(19) United States
(12) Patent Application Publication
(10) Pub. No.: US 2007/0148105 A1
(43) Pub. Date: Jun. 28, 2007

(54) Compositions and methods comprising magnetic particles for health use

(76) Inventor: Donald Spector, New York, NY (US)

Correspondence Address: Glen M. Diehl;NORTON & DIEHL LLC, Suite 110, 77 Brant Ave., Clark, NJ, 07066, US

(21) Appl. No.: 11/318,191

(22) Filed: Dec. 22, 2005

(51) Int. Cl. A61K 33/26; A61K 9/127; A61K 9/14; A61K 8/19

(52) U.S. Cl. 424/59; 424/450; 424/489; 424/646

(57) ABSTRACT

The present invention provides a topical composition comprising a topical composition base and a plurality of magnetic particles. The topical composition can further comprise one or more active agents. The magnetic particles and/or active agent(s) can optionally be encapsulated and/or coated. Methods for making and using the topical composition are also provided.

### CROSS-REFERENCE TO RELATED APPLICATIONS

This application claims the benefit of U.S. Provisional patent application Ser. No. ______, filed ______, which is hereby incorporated by reference.

### BACKGROUND OF THE INVENTION

There has been considerable research showing that magnetic fields have a positive influence on health. Many of these studies have now been reported in legitimate medical journals and are used in treatments for arthritis, wellness and other specific ailments. For example, Eccles concluded that “weight of evidence from published, well-conducted controlled trials suggests that static magnetic fields are able to induce analgesia.” J. Altern. Complement. Med., 11 (3):495-509 (2005). Similarly, Weintraub et al. reported that “constant wearing of multipolar, static magnetic (450G) shoe insoles helps to alleviate pain associated with symptomatic diabetic peripheral neuropathy.” Arch. Phys. Med. Rehabil. 84(5):736-46 (2003).

The market for bracelets and other jewelry has soared as a greater number in the population believe in magnetic theory for healing and health. However, these magnets are placed over a very small area of the body and they rely on force fields to carry them, which are non-specific in design.

Accordingly, new and improved compositions and method of using magnetic fields are needed.

### SUMMARY OF THE INVENTION

In one aspect, the present invention provides a topical composition comprising a topical composition base and a plurality of magnetic particles. The topical composition may also comprise one or more active agents selected from the group consisting of vitamins, antimicrobial agents, anti-inflammatory agents, soothing agents, skin lightening agents, bronzers, anti-wrinkle agents, sunscreen agents, anti-itching agents, antioxidants, fragrances, conditioners, heating agents, cooling agents, and any combination thereof. The magnetic particles and/or active agent(s) can be encapsulated, coated and/or packaged in controlled-release formulations.

In another aspect, the present invention provides for a method of treating a mammal with a condition treatable by an application of a magnetic field, such as pain, comprising applying to the desired area of said mammal a topical composition comprising a topical composition base and a plurality of magnetic particles. Preferably, the mammal is a human.

In yet another aspect, the present invention provides for a method of making a topical composition comprising mixing a topical composition base with a plurality of magnetic particles and, optionally, one or more active agents under conditions sufficient to form the topical composition. The magnetic particles can be magnetized prior to, during or after mixing with the base.

### BRIEF DESCRIPTION OF THE DRAWINGS

FIG. 1 illustrates a topical cream composition comprising encapsulated magnetic particles and a plurality of encapsulated active ingredients.

### DETAILED DESCRIPTION OF THE INVENTION

The present invention solves the shortcomings of magnetic jewelry by incorporating small magnetic particles into cosmetic or pharmaceutical topical compositions. Thus, the magnetic fields can then be applied to a more specific surface area thus better serving the needs of the user than, for example, wearing an anklet or a bracelet. When worn, the magnetic fields will be generated directly on the surface of the skin, therefore improving the application of the field to a subject, which is important because the fields diminish rapidly with distance (according to an inverse square of the distance).

One aspect of the present invention provides a topical composition comprising a topical composition base and a plurality of magnetic particles. Non-limiting examples of materials suitable for the magnetic particles include iron, Fe₂O₃, other ferromagnetic elements and compositions, nickel, cobalt, neodymium, samarium or any mixture thereof.

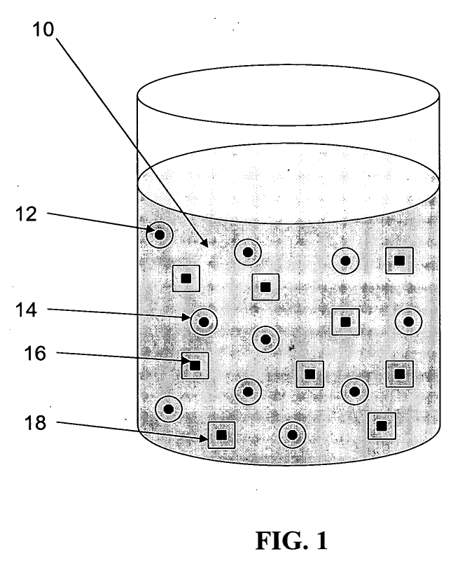

FIG. 1 illustrates one embodiment of this aspect of the present invention. A cream base 10 is provided. A plurality of magnetic particles 12 are mixed into the cream base. The plurality of magnetic particles can be encapsulated with an encapsulant 14. One or more active agents 16 can also be mixed into the cream base 10. The one or more active agents 16 can also be encapsulated with an encapsulant 18. Further, the cream base 10 can be a liquid. The magnetic particles preferably comprise from about 0.001% to about 5% w/w of the topical composition.

The topical composition can be in a solid or liquid form. Suitable solid topical compositions include, for example, powders, sticks or bars similar to deodorant sticks. Suitable liquid topical compositions include, for example, gels, dispersions, microemulsions, emulsions, suspensions, foams, mousses, pastes, creams, balms, ointments, body washes, rinses, lotions, oils and sprays.

A person of ordinary skill in the art will appreciate that the topical compositions of the present invention may be formulated with emollients, solvents, thickeners, humectants and/or powders. Suitable emollients include, but are not limited to, stearyl alcohol, mink oil, cetyl alcohol, oleyl alcohol, isopropyl laurate, polyethylene glycol, olive oil, petroleum jelly, palmitic acid, oleic acid, and myristyl myristate.

Suitable solvents include, but are not limited to, ethyl alcohol, isopropyl alcohol, acetone, diethylene glycol, ethylene glycol, dimethyl sulfoxide, and dimethyl formamide.

Suitable humectants include, but are not limited to, acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxy polyethylene glycol, myristamidobutyl guanidine acetate, polyglyceryl sorbitol, potassium pyrollidone carboxylic acid (PCA), propylene glycol, sodium pyrollidone carboxylic acid (PCA), sorbitol, sucrose, and urea. Other humectants may be used for yet additional embodiments of this invention, as will be appreciated by those skilled in the art.

Suitable thickeners include, but are not limited to, polysaccharides, in particular xantham gum, guar-guar, agar-agar, alginates, carboxymethylcellulose, relatively high molecular weight polyethylene glycol mono- and diesters of fatty acids, polyacrylates, polyvinyl alcohol and polyvinylpyrrolidone, surfactants such as, for example, ethoxylated fatty acid glycerides, esters of fatty acids with polyols such as, for example, pentaerythritol or trimethylpropane, fatty alcohol ethoxylates or alkyl oligoglucosides, and electrolytes, such as sodium chloride and ammonium chloride.

The compositions of the present invention may also include components that enhance the penetration of the magnetic particles into the skin and/or to the site of action. Suitable examples of penetration enhancers include, but are not limited to, urea, propan-2-ol, polyoxyethylene ethers, terpenes, cis-fatty acids (oleic acid, palmitoleic acid), acetone, laurocapram dimethyl sulfoxide, 2-pyrrolidone, oleyl alcohol, glyceryl-3-stearate, cholesterol, myristic acid isopropyl ester, and propylene glycol.

The methods of formulation of different topical compositions are well known to a skilled artisan. For example, the base of the topical described in the instant disclosure may be formulated such as a cream, an ointment, an aerosol formulation, a non-aerosol spray, a gel, a foam, a solution, a suspension, a dispersion, an emulsion, a microemulsion, a paste, a powder, a solid stick (e.g., wax- or petroleum-based sticks), a wipe, an oil, a lotion, and the like. For administration, the cosmetic and dermatological preparations of the invention may be applied to the skin in adequate quantity in the manner conventional for cosmetics. A person skilled in the art can select the appropriate presentation form, and also the method for preparing it, on the basis of his general knowledge, taking into account on the one hand the nature of the constituents used, and on the other hand the intended use of the composition.

In some embodiments, in addition to the plurality of magnetic particles, one or more active agents are included into the topical composition. The magnetic particles and the second active(s) agent may perform the same or different functions. For example, the designer of the product can use vitamins, antimicrobial agents, anti-inflammatory agents, non-steroidal cosmetic soothing agents, skin lightening agents, anti-wrinkle agents, sunscreen agents, anti-itching agents, antioxidants, fragrances, conditioners, heating agents, cooling agents, bronzers, and other agents or any combination thereof. The active agent(s) preferably comprise from about 0.001% to about 10% w/w of the topical composition.

Non-limiting examples of vitamins useful herein include, but are not limited to, vitamin A, vitamin B₁-B₁₂, biotin, vitamin C, pantothenic acid, vitamin K, vitamin D, vitamin E and mixtures thereof.

Non-limiting examples of antimicrobial agents useful herein include, but are not limited to, beta-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione; clotrimazole; alantolactone; isoalantolactone; alkanet extract (alaninin); anise; arnica extract (helenalin acetate and 11, 13 dihydrohelenalin); Aspidium extract (phloro, lucinol containing extract); barberry extract (berberine chloride); bay sweet extract; bayberry bark extract (myricitrin); benzalkonium chloride; benzethonium chloride; benzoic acid and its salts; benzoin; benzyl alcohol; blessed thistle; bletilla tuber; bloodroot; bois de rose oil; burdock; butyl paraben; cade oil; CAE (available from Ajinomoto, located in Teaneck, N.J.); cajeput oil; Cangzhu; capsicum frutescens extract; caraway oil; cascarilla bark (sold under the tradename ESSENTIAL OIL); cedarleaf oil; chamomille; chaparral; chlorhexidine gluconate; chlorophenesin; chlorxylenol; cinnamon oil; citronella oil; clove oil; Crinipan AD (available from Climbazole); 2,3-dihydro-farnesol; dehydroacetic acid and its salts; dill seed oil; DOWICIL 200 (available from Dow Chemical, located in Midland, Mich.); echinacea; elenolic acid; epimedium; ethyl paraben; Fo-Ti; galbanum; garden bumet; GERMALL 115 and GERMALL II (available from ISP-Sutton Labs, located in Wayne, N.J.); German chamomile oil; giant knotweed; GLYDANT (available from Lonza, located in Fairlawn, N.J.); GLYDANT PLUS (available from Lonza); grapefruit seed oil; 1,6 hexanediol; hexamidine diisethionate; hinokitiol; honey; honeysuckle flower; hops; immortelle; iodopropynl butyl carbamide (available from Lonza); isobutyl paraben; isopropyl paraben; JM ACTICARE (available from Microbial Systems International, located in Nottingham, NG); juniper berries; KATHON CG (available from Rohm and Haas, located in Philadelphia, Pa.); kojic acid; labdanum; lavender; lemon balm oil; lemon grass; methyl paraben; mint; mume; mustard; myrrh; neem seed oil; ortho phenyl phenol; olive leaf extract (available from Bio Botanica); parsley; patchouly oil; peony root; 1,2 pentandiol; PHENONIP (available from Nipa Labs, located in Wilmington, Del.); phenoxyethanol; phytosphingosine; pine needle oil; PLANSERVATIVE (available from Campo Research); propyl paraben; purslane; quillaira; rhubarb; rose geranium oil; rosemary; sage; salicylic acid; sassafras; savory; sichuan lovage; sodium meta bisulfite; sodium sulfite; SOPHOLIANCE (available from Soliance, located in Compiegne, France); sorbic acid and its salts; sphingosine; stevia; storax; sucrose esters; tarmic acid; tea; tea tree oil (cajeput oil); thyme; triclosan; triclocarban; tropolone; turpentine; umbelliferone (antifungal); yucca; and mixtures thereof.

Non-limiting examples of anti-inflammatory agents useful herein include, but are not limited to, hydrocortisone, non-steroidal anti-inflammatory agents such as oxicans, salicylates, acetic acid derivatives, fenamates, propionic acid derivatives, pyrazoles, substituted phenyl compounds, 2-naphthyl containing compounds, and natural anti-inflammatory agents such as aloe vera. Examples of anti-inflammatory agents are described in U.S. Pat. No. 5,487,884, the entire contents of which is incorporated herein by reference.

Non-limiting examples of useful cosmetic soothing agents useful herein include, but are not limited to, acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, absinthium, acacia, aescin, alder buckthorn extract, allantoin, aloe, APT (available from Centerchem), amica, astragalus, astragalus root extract, azulene, Baicalin SR 15 (available from Barnet Products Dist.), baikal skullcap, baizhu, balsam canada, bee pollen, BIOPHYTEX (available from Laboratories Serobiologiques), bisabolol, black cohosh, black cohosh extract blue cohosh, blue cohosh extract, boneset, borage, borage oil, bradykinin antagonists, bromelain, calendula, calendula extract, Canadian Willowbark Extract (available from Fytokem), candelilla wax, Cangzhu, canola phytosterols, capsicum, carboxypeptidase, celery seed, celery stem extract, CENTAURIUM (available from Sederma), centaury extract, chamazulene, chamomile, chamomile extract, chaparral, chaste tree, chaste tree extract, chickweed, chicory root, chicory root extract, chirata, chishao, collodial oatmeal, comfrey, comfrey extract, CROMOIST CM GLUCAN (available from Croda), darutoside, dehurian angelica, devil's claw, divalent metals (such as, magnesium, strontium, and manganese), doggrass, dogwood, Eashave (available from Pentapharm), eleuthero, ELHIBIN (available from Pentapharm), ENTELINE 2 (available from Secma), ephedra, epimedium, esculoside; ethacrynic acid, evening primrose, eyebright, Extract LE-100 (available from Sino Lion), Fangfeng, feverfew, ficin, forsythia fruit, Fytosterol 85 (available from Fytokem), ganoderma, gaoben, Gatuline A (available from Gattefosse), gentian, germanium extract, gingko bilboa extract, ginkgo, ginseng extract, goldenseal, gorgonian extract, gotu kola, grape fruit extract, guaiac wood oil, guggal extract, helenalin esters, henna, honeysuckle flower, horehound extract, horsechestnut, horsetail, huzhang, hypericum, ichthyol, immortelle, ipecac, job's tears, jujube, kola extract, LANACHRYS 28 (available from Lana Tech), lemon oil, lianqiao, licorice root, ligusticum, ligustrum, lovage root, luffa, mace, magnolia flower, manjistha extract, margaspidin, matricin, melatonin, MICROAT IRC (available from Nurture), mints, mistletoe, Modulene (available from Seporga), mono or diglucosides of glabridin, mono or diglucosides of gentisin, MTA (5′-deoxy-5′-methythioadenosine), mung bean extract, musk, N-methyl arginine, oat beta glucan, oat extract, orange, panthenol, papain, phenoxyacetic acid, peony bark, peony root, Phytoplenolin (available from Bio Botanica), phytosphingosine, Preregen (available from Pentapharm), purslane, QUENCH T (available from Centerchem), quillaia, red sage, rehmannia, rhubarb, rosemary, rosmarinic acid, royal jelly, rue, rutin, sandlewood, sanqi, sarsaparilla, saw palmetto, SENSILINE (available from Silab), SIEGESBECKIA (available from Sederma), stearyl glycyrrhetinate, Stimutex (available from Pentapharm), storax, strontium nitrate, sweet birch oil, sweet woodruff, tagetes, tea extract, thyme extract, tienchi ginseng, tocopherol, tocopheryl acetate, triclosan, turmeric, urimei, ursolic acid, white pine bark, witch hazel xinyi, yarrow, yeast extract, yucca, and mixtures thereof.

Non-limiting examples of skin lightening agents useful herein include, but are not limited to, adapalene, aloe extract, alpha-glycaryl-L-ascorbic acid, aminotyroxine, ammonium lactate, anethole derivatives, apple extract, arbutin, areca catechu L. extract, ascorbic acid, ascorbyl palmitate, azelaic acid, bamboo extract, bearberry extract, bletilla tuber, bupleurum falcatum extract, burnet extract, Burnet Power (available from Barnet Products), butyl hydroxy anisole, butyl hydroxy toluene, butyl resoreinol, Chuanxiong, cola decaballo extract, Dang-Gui, deoxyarbutin, 1,3 diphenyl propane derivatives, 2,5 dihydroxybenzoic acid and its derivatives, 2-(4-acetoxyphenyl)-1,3 dithane, 2-(4-hydroxyphenyl)-1,3 dithane, ellagic acid, escinol, estragole derivatives, esculoside, esculetin, FADEOUT (available from Pentapharm), Fangfeng, fennel extract, gallic acid and its derivatives, ganodenna extract, gaoben, GATULINE WHITENING (available from Gattlefosse), genistic acid and its derivatives, gentisyl alcohol, glabridin and its derivatives, gluco pyranosyl-1-ascorbate, gluconic acid, glucosamine, glycolic acid, glycyrrhizinic acid, green tea extract, 4-Hydroxy-5-methyl-3[2H]-furanone, hydroquinine, 4 hydroxyanisole and its derivatives, 4-hydroxy benzoic acid derivatives, hydroxycaprylic acid, hyptis extract, inositol ascorbate, kojic acid, kojic dipalnitate, lactic acid, lemon extract, licorice extract, Licorice P-TH (available from Barnet Products), linoleic acid, magnesium ascorbyl phosphate, Melfade (available from Pentapharm), MELAWHITE (available from Pentapharm), Melanostatine DM (available from Laboratories Seporga), morus alba extract, mulberry root extract, niacinamide, 5-octanoyl salicylic acid, parsley extract, phellinus linteus extract, pinon blanco extract, pinon negro extract, piri-piri extract, pyrogallol derivatives, retinoic acid, retinol, retinyl esters (acetate, propionate, palmitate, linoleate), 2,4 resorcinol derivatives, 3,5 resorcinol derivatives, rose fruit extract, rucinol, salicylic acid, Song-Yi extract, Sophora Powder (available from Barnet Products), 4-thioresorein, 3,4,5 trihydroxybenzyl derivatives, tranexamic acid, tyrostat (Rumex Extract available from Fytokem), Tyroslat 10,11 (available from Fytokem), vanilla derivatives, vitamin D₃ and its analogs, and mixtures thereof.

Non-limiting examples of suitable sunscreening agents useful herein include, but are not limited to, p-aminobenzoic acid, its salts and derivatives, anthranilates, salicylates, cinnamic acid derivatives, dihydroxycinnamic acid derivatives, trihydroxycinnamic acid derivatives, hydrocarbons, dibenzalacetone and benzalacetophenone, naphthosulfonates, dihydroxy-naphthoic acid and its salts, o- and p-hydroxy-biphenyldisulfonates, coumarin derivatives, diazoles quinine salts, quinoline derivatives, hydroxy or methoxy substituted benzophenones, uric and vilouric acids, tannic acid and its derivatives, hydroquinone, benzophenones, 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Still other useful sunscreens are those disclosed in U.S. Pat. Nos. 4,937,370 and 4,999,186, each of which is incorporated by reference herein in its entirety. Still other useful sunscreens include, but are not limited to, aminobenzoic acid (PABA), benzylidene camphor, butyl methoxy dibenzoyl methane, diethanolamine p-methoxycinnamate, dioxybenzone, ethyl dihydroxypropyl (PABA), glyceryl aminobenzoate, homomenthyl salicylate, isopropyl dibenzoyl methane, lawsone and dihydroxyacetone, menthyl anthranilate, methyl anthranilate, methyl benzylidene camphor, octocrylene, octyl dimethyl (PABA), octyl methoxycinnamate, oxybenzone, 2-phenylbenzimidazole-5-sulfonic acid, red petrolatum, sulisobenzone, titanium dioxide, triethanolamine salicylate, zinc oxide, and mixtures thereof Especially preferred examples of these sunscreens include those selected from the group consisting of 4-N,N-(2-ethylhexyl)methylaminobenz-oic acid ester of 2,4-dihydroxybenzophenone, 4-N,N-(2-ethylhexyl)methylami-nobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylami-nobenzoic acid ester of 4-(2-hydroxyethoxy)dibenzoylmethane, and mixtures thereof.

Non-limiting examples of anti-itch ingredients useful herein include, but are not limited to, Stimu-tex (available from Pentapharm); Takanal (available from Ikeda-Distributer); Ichthyol (available from International Sourcing-Distributor); Oxygenated Glyceryl Triesters (available from Seporgia) and mixtures thereof.

Non-limiting example of antioxidants useful herein include, but are not limited to, vitamin E, tocopheryl acetate, betaglucan, coenzyme Q10, butylated hydroxy toluene (BHT), butylated hydroxy anisole BHA, superoxide dismutose, propylgallate, and the like.

Non-limiting examples of skin conditioners useful herein include, but are not limited to, mineral oil, petrolatum, vegetable oils (such as soybean or maleated soybean oil), dimethicone, dimethicone copolyol, cationic monomers and polymers (such as guar hydroxypropyl trimonium chloride and distearyl dimethyl ammonium chloride) as well as combinations thereof. Illustrative moisturizers are polyols such as sorbitol, glycerin, propylene glycol, ethylene glycol, polyethylene glycol, polypropylene glycol, 1,3-butane diol, hexylene glycol, isoprene glycol, xylitol, fructose and mixtures thereof.

Suitable fragrances which can be used in the present invention comprise, for example, the high boiling components of woody/earthy bases containing exotic materials such as sandalwood oil, civet, patchouli oil, and the like. The fragrances can be of a light, floral fragrance, such as for example, high boiling components of rose extract, violet extract, and the like. The fragrances can be formulated to provide desirable fruity odors, such as for example lime, lemon, orange, and the like. The fragrance can be any material of appropriate chemical and physical properties which exudes a pleasant or otherwise desirable odor when applied to skin. Materials suitable for use in the present invention are described more fully in S. Arctander, Perfume Flavors and Chemicals, Vols. I and II, Aurthor, Montclair, N.J. and the Merck Index, 8th Edition, Merck & Co., Inc. Rahway, N.J., each of which is incorporated herein by reference in it entirety.

Suitable non-limiting examples of skin heating agents useful herein include, but are not limited to, polyvinyl amine, polyalkyleneamine or polyalkyleneimine, as described in U.S. Patent Publication No. 20040253198, which is incorporated by reference herein in its entirety.

Suitable non-limiting examples of skin cooling agents useful herein include, but are not limited to, menthol, menthol esters, methyl salicylate, camphor, benzocaine, dibucaine, dyclonine, lidocaine, pramoxine, tetracaine, ephedrine, epinephrine, phenylephrine, and their derivatives, and combinations thereof as described in the U.S. Patent Publication 20040175340, which is incorporated herein by reference in its entirety.

Non-limiting examples of bronzers useful herein include, but are not limited to, juglone, lawsone, and Caymen Sun Secrets® Skin Bronzer.

In some embodiments, the magnetic particles and/or the active agent(s) are encapsulated before inclusion in the topical composition. Suitable encapsulating compounds and techniques of encapsulation of compounds for topical compositions are well known in the art and described, for example, in U.S. Pat. Nos. 4,478,822, 4,803,195, 4,917,982, and 5,861,440, each of which is incorporated by reference herein in its entirety. Such encapsulation agents include, for example, microspheres, microparticles, microbubbles and liposomes.

The active agent(s) may also be delivered in a controlled-release form, such as, for example, microspheres and/or nanospheres as described for example, in U.S. Pat. No. 6,589,562 and U.S. Patent Publication Nos. 20030198652 and 20030232091, each of which is incorporated herein by reference in its entirety. It is possible to design the encapsulation mode and to select encapsulants in such a way that they will react to environmental changes and release the packaged compounds. For example, if it is desirable to create a system wherein the release of a compound encapsulated in a microsphere is triggered by increased moisture, the following materials would be suitable for the microsphere: water soluble and water dispersible synthetic polymers and copolymers, starch derivatives, polysaccharides, hydrocolloids, natural gums, proteins, and mixtures thereof. If the changes in the pH are used for triggering the release of the encapsulated compounds, a skilled artisan may use pH-sensitive substrates, such as, for example, polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

In yet other embodiments, the magnetic particles of the topical composition may additionally, or alternatively, be coated with a coating that optionally comprises a tinting agent. Suitable coating materials include, for example, the same coloration and pigment materials used in the normal practice of makeup, thereby not appearing discontinuous with normal foundation makeup. Suitable tinting agents include, for example, zinc oxide, kaolin, mica, iron oxide, titanium dioxide (U.S. Pat. No. 6,022,532), anthraquinone compounds (U.S. Pat. No. 6,660,284) and octinoxate.

The methods of mixing magnetic particles and, optionally, active agent(s) into a topical composition base are within the expertise of a person of ordinary skill in the art. In general, a topical composition base is mixed with a plurality of magnetic particles and, optionally, one or more active agents under conditions sufficient to form the topical composition. If the magnetic particles and/or active agents are encapsulated or coated, such encapsulation or coating is preferably done prior to mixing with topical composition base. If desired, the particles can be magnetized in a suitable container (e.g., bottle) during or following mixing with the topical composition base.

The topical composition of the present invention is useful for treatment of conditions treatable with an application of magnetic field. Depending on the form of the topical composition selected by the user, the user would rub or spray this composition onto a desired skin area. For example, if the user has pain associated with diabetic neuropathy, she can apply the topical composition of the present invention to her feet. In this non-limiting example, the topical composition of the present invention would be especially suitable for applications in situations where the user cannot or would not wear shoes with magnetic soles, such as, for example, in bed.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A topical composition comprising a topical composition base and a plurality of encapsulated magnetic particles.

2. The topical composition of claim 1, wherein the magnetic particles are selected from the group consisting of iron, Fe₂O₃, other ferromagnetic elements and compositions, nickel, cobalt, neodymium, samarium and combinations thereof.

3. The topical composition of claim 1, wherein the magnetic particles comprise from about 0.001% to 5% w/w of said topical composition.

4. The topical composition of claim 1, wherein the magnetic particles are encapsulated within an encapsulant selected from the group consisting of microspheres, microparticles, microbubbles and liposomes.

5. The topical composition of claim 1, further comprising one or more active agents independently selected from the group consisting of vitamins, antimicrobial agents, anti-inflammatory agents, soothing agents, skin lightening agents, bronzers, anti-wrinkle agents, sunscreen agents, anti-itching agents, antioxidants, fragrances, conditioners, heating agents, cooling agents and combination thereof.

6. The topical composition of claim 5, wherein the active agent or agents comprises from about 0.001% to about 10% w/w of said topical composition.

7. The topical composition of claim 5, wherein the active agent or agents is encapsulated.

8. The topical composition of claim 7, wherein the active agent or agents is encapsulated in an encapsulant selected from the group consisting of microspheres, microparticles, microbubbles and liposomes.

9. The topical composition of claim 1, wherein the composition is in a solid form.

10. The topical composition of claim 9, wherein the composition is in a solid form selected from the group consisting of sticks, bars and powders.

11. The topical composition of claim 1, wherein the composition is in a liquid form.

12. The topical composition of claim 11, wherein the composition is in a liquid form selected from the group consisting of gels, dispersions, emulsions, microemulsions, suspensions, foams, mousses, pastes, creams, balms, ointments, body washes, rinses, lotions, oils and sprays.

13. A method of treating a mammal having a condition treatable by magnetic therapy comprising applying to a desired area of said mammal a topical composition comprising a topical composition base and a plurality of encapsulated magnetic particles.

14. The method of claim 13, wherein the mammal is a human.

15. The method of claim 13, wherein the condition is pain.

16. The method of claim 13, wherein the magnetic particles are encapsulated in an encapsulant selected from the group consisting of microspheres, microparticles, microbubbles and liposomes.

17. The method of claim 13, wherein the topical composition further comprises one or more active agents independently selected from the group consisting of vitamins, antimicrobial agents, anti-inflammatory agents, soothing agents, skin lightening agents, bronzers, anti-wrinkle agents, sunscreen agents, anti-itching agents, antioxidants, fragrances, conditioners, heating agents, cooling agents and combination thereof.

18. The method of claim 17, wherein the active agent or agents is encapsulated.

19. The method of claim 18, wherein the active agent or agents is encapsulated in an encapsulant selected from the group consisting of microspheres, microparticles, microbubbles and liposomes.

20. A method of making a topical composition comprising mixing a topical composition base with a plurality of encapsulated magnetic particles under conditions sufficient to form said topical composition.

21. The method of claim 20, further comprising mixing with the topical composition base one or more active agents independently selected from the group consisting of vitamins, antimicrobial agents, anti-inflammatory agents, soothing agents, skin lightening agents, bronzers, anti-wrinkle agents, sunscreen agents, anti-itching agents, antioxidants, fragrances, conditioners, heating agents, cooling agents and combinations thereof.

22. The method of claim 20, wherein the magnetic particles are magnetized prior to mixing with the topical composition base.

23. The method of claim 20, wherein the magnetic particles are magnetized during or after mixing with the topical composition base.

24. A topical composition comprising a topical composition base and a plurality of coated magnetic particles.

25. The topical composition of claim 24, wherein magnetic particles are coated with a coating having a coloration continuous with normal foundation makeup.

26. The topical composition of claim 24, wherein the coating comprises a tinting agent.
